(19) United States
(12) Patent Application Publication
(10) Pub. No.: US 2008/0268488 A1
(43) Pub. Date: Oct. 30, 2008

(54) Screening Method for Dnak Inhibitors

(76) Inventor: Michael A. Sturgess, Quakertown, PA (US)

Correspondence Address: FOX ROTHSCHILD LLP;PRINCETON PIKE CORPORATE CENTER, 2000 Market Street, Tenth Floor, Philadelphia, PA, 19103, US

(21) Appl. No.: 12/093,411

(22) PCT Filed: Nov. 6, 2006
(86) PCT No.: PCT/US06/43174

§ 371 Date: May 12, 2008

Related U.S. Application Data
(60) Provisional application No. 60/735,907, filed on Nov. 10, 2005.

(51) Int. Cl. C12Q 1/26

(52) U.S. Cl. 435/25

(57) ABSTRACT

Screening methods for identification of inhibitors of DnaK activity are provided. Such inhibitors have utility as antibacterial agents.

### FIELD OF THE INVENTION

The invention relates generally to DnaK inhibitors. More specifically, the invention relates to screening methods for identification of compounds that inhibit DnaK for use as antibacterial agents.

### BACKGROUND OF THE INVENTION

The continuing search for new and effective antibacterial agents that can treat infections caused by organisms that are increasingly resistant to known classes of antibacterial agents has identified a plethora of potential next generation antibiotics. Many of these agents have subsequently been shown to demonstrate either poor physiochemical properties, an increased tendency to induce bacterial resistance, poor toxicological profiles, or low efficacy in vivo. Over the past decade or so, certain antibacterial peptides and glycopeptides isolated from insects have been noted as promising candidates for drug development programs (see, e.g., Hultmark, Trends Genet., 9:178-183 (1993); Gillespie et al., Annu. Rev. Entomol., 42:611-643 (1997); Otvos, Jr. et al., Protein Sci. 9:742-749 (2000); International Patent Publication No. WO 94/05787, published Mar. 17, 1994; French Patent No. 2733237, granted Oct. 25, 1996; International Patent Publication No. WO 99/05270, published Feb. 4, 1999; International Patent Publication No. WO 97/30082, published Aug. 21, 1997; French Patent No. 2695392, granted Mar. 11, 1994; French Patent No. 2732345, granted Oct. 4, 1996; and International Patent Publication No. WO 00/78956, published Dec. 28, 2000.)

While many antibacterial peptides from other origins kill bacteria by disrupting the cell membrane or cell wall of the bacteria, a subset of the insect-derived antibacterial peptides have an unusual mode of action, i.e., they inhibit the bacterial chaperone protein DnaK. Two such peptides are drosocin, a 19 amino acid residue peptide from Drosophila (Bulet et al., J. Biol. Chem. 268:14893-14897 (1993)) and pyrrhocoricin, a 20 amino acid residue peptide from Pyrrhocoris (Cociancich et al., Biochem. J. 300:567-575 (1994)). Drosocin and pyrrhocoricin are glycopeptides characterized by the presence of a disaccharide in the mid-chain position. The presence of the disaccharide increases the in vitro antibacterial activity of drosocin, but decreases the activity of pyrrhocoricin (Bulet et al., supra; Hoffmann et al., Biochim. Biophys. Acta, 1426:459-467 (1999)).

While active in vitro, both drosocin and pyrrhocoricin are known to be highly susceptible to proteolytic degradation in the presence of mammalian serum. Both aminopeptidase and carboxypeptidase cleavage products are observed. Metabolites lacking as few as five amino terminal or two carboxy terminal amino acids have been shown to be inactive as antibacterial agents in vitro (Bulet et al., supra; Hoffmann et al., supra).

The interaction of non-glycosylated pyrrhocoricin with bacterial DnaK has been extensively studied (see, e.g., Otvos, Jr. et al., Biochemistry 39:14150-14159 (2000); Kragol et al., Biochemistry 40:3016-3026 (2001)). Residues within the N-terminal half of the peptide have been implicated in binding to DnaK, and were, shown to specifically interact with helices D and E of the helical lid of the bacterial protein (Kragol et al., Eur. J. Biochem. 269:4226-4237 (2002)).

DnaK has been demonstrated to be the central protein in a multiprotein bacterial chaperone system including the chaperone protein DnaK and a variety of co-chaperone proteins such as DnaJ and GrpE. The co-chaperone proteins are essential to the efficient physiological processing of both natural and unnatural substrates. One role for this chaperone system is to catalyze the refolding of either unfolded or misfolded bacterial proteins, as is evident from the role of this system in the heat-shock response. An additional role of the DnaK chaperone system is the regulation of gene expression through the processing of specific RNA polymerase subunits.

DnaK deletion mutants of many organisms have been generated and overall the mutant strains have been shown to exhibit lower growth rates, greater susceptibility to environmental stress, reduced viability in cellular environments, and reduced ability to establish infections in vivo compared to the wild-type strains.

With the role of the DnaK chaperone system in bacterial growth and survival, as well as the validation of DnaK as an antibacterial target, firmly established, there is a need for new DnaK inhibitors and methods for their identification.

### SUMMARY OF THE INVENTION

Applicants have invented screening methods for identification of inhibitors of DnaK activity. Such inhibitors have utility as antibacterial agents.

Accordingly, the present invention provides a screening method for identification of a DnaK inhibitor, comprising the steps of:
(a) providing a denatured substrate protein in solution;
(b) contacting said substrate protein with DnaK, or a homolog thereof, in the presence and absence of a compound suspecting of having DnaK inhibitory activity; and
(c) determining whether the activity of said substrate protein in the presence of said compound is decreased relative to the activity of said substrate protein in the absence of said compound,
   whereby a decrease in substrate protein activity indicates that said compound is a DnaK inhibitor.

In some embodiments, positive and/or negative controls for substrate protein refolding and/or DnaK inhibition are provided in the screening method. Known DnaK inhibitors useful in the screening methods of the present invention include, e.g., pyrrhocoricin, drosocin and analogs thereof. Co-chaperone proteins, such as, e.g., DnaJ and GrpE, can also be included in the screening method to facilitate substrate protein refolding.

### BRIEF DESCRIPTION OF THE DRAWINGS

The accompanying drawings, which are incorporated in and form a part of the specification, illustrate non-limiting embodiments of the present invention, and together with the description serve to explain the principles of the invention.

FIG. 1 shows the structure of the pyrrhocoricin analog CHP-105, a peptide inhibitor of E. coli DnaK.

FIG. 2 shows the dose response of inhibition of DnaK-induced refolding of firefly luciferase by CHP-105 in the presence of BSA and DTT.

FIG. 3 shows the effect of DMSO on inhibition of DnaK-induced refolding of firefly luciferase by CHP-105.

### DETAILED DESCRIPTION OF THE INVENTION

The present invention relates to screening methods for identification of inhibitors of DnaK activity. The present in vitro screening method is based on the ability of DnaK to induce refolding of a denatured substrate protein.

Compounds are screened for their ability to inhibit DnaK-induced substrate protein refolding.

Any protein with a detectable activity (i.e., “reporter activity”) can be used as the substrate protein, including e.g., β-galactosidase, chloramphenicol acetyltransferase, alkaline phosphatase, green fluorescent protein, Renilla luciferase and firefly luciferase. Firefly luciferase is preferred because its activity is easily detected and quantified using commercially available kits, and thus is easily adaptable to a high-throughput screening (“HTS”) format.

When using firefly luciferase, denaturation is preferably performed chemically, e.g., in Tris-buffered 8 M urea or 6 M guanidinium hydrochloride, pH 7.4, for between about 30 min to 90 min at room temperature. DTT (e.g., 5 mM) is also typically included in the denaturation reaction. Chemically denatured firefly luciferase is well characterized and has been shown to be a substrate for the DnaK chaperone system (see, e.g., Szabo et al., Proc. Natl. Acad. Sci. USA 91:10345-10349 (1994)).

The denatured substrate protein is then refolded by contacting it with DnaK. The concentration of DnaK used in the refolding reaction is preferably below 1 μM, more preferably below 500 mM and even more preferably below 250 Nm. The refolding reaction is typically initiated by diluting the substrate protein from about 50-fold to about 500-fold in a buffered salt solution (e.g., 10 mM MOPS, pH 7.5, 50 mM KCl, 5 mM MgCl₂) containing the DnaK and an energy source, typically ATP (e.g., 2 mM), for between about 60 to about 300 min at room temperature. DnaK can be omitted from the refolding reaction to serve as a negative control for spontaneous refolding.

The DnaK, or a homolog thereof, can be purified or cloned from any source of interest, including e.g., Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, Bordetella and Francisella, or can be commercially obtained from, e.g., Stressgen Bioreagents Corp., Vancouver, B.C. Co-chaperone proteins, such as DnaJ and GrpE, or analogs thereof, are preferably included in the reaction to enhance refolding. When DnaJ and GrpE are included in the refolding reaction, the concentration of GrpE is preferably greater than that of DnaK and DnaJ, preferably at least about 2-fold greater than each.

The DnaK refolding reaction is performed in the presence and absence of a compound suspecting of having DnaK inhibitory activity (i.e., “candidate agent”). Such compounds are typically provided as a library. Candidate agents encompass numerous chemical classes, though typically they are organic molecules, preferably small organic compounds having a molecular weight of more than 50 and less than about 2,500 daltons. Such “small molecules” are typically suspended in an organic solvent, such as, e.g., DMSO, prior to their inclusion in the folding reaction.

Candidate agents will typically comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including, but not limited to, peptides, proteins, saccharides, fatty acids, steroids, purines, pyrimidines, and various derivatives, structural analogs and combinations thereof.

Additionally, natural or synthetically produced libraries and compounds can be readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

The effect of the candidate agent on DnaK-mediated refolding is determined by measuring the activity of any refolded substrate protein. For example, if firefly luciferase is employed as the substrate protein, luciferase activity can be detected and measured using the appropriate instrumentation and reagents, for example, by detecting light emission using a luminometer upon addition of luciferin. Reagents and kits for measuring luciferase activity are commercially available from e.g., Promega, Madison, Wis. (e.g., “Steady-Glo®”). If the activity of the substrate protein in the presence of the candidate agent is decreased relative to the activity of the substrate protein in the absence of the agent, the agent is considered a DnaK inhibitor. The compounds thus identified can be used directly or serve as conventional “lead compounds” for the development of antibacterial agents.

Generally, a plurality of assays is performed in parallel with different agent concentrations to obtain a differential response to the various concentrations. Typically, one of these concentrations serves as a negative control, i.e., at zero concentration or below the level of detection.

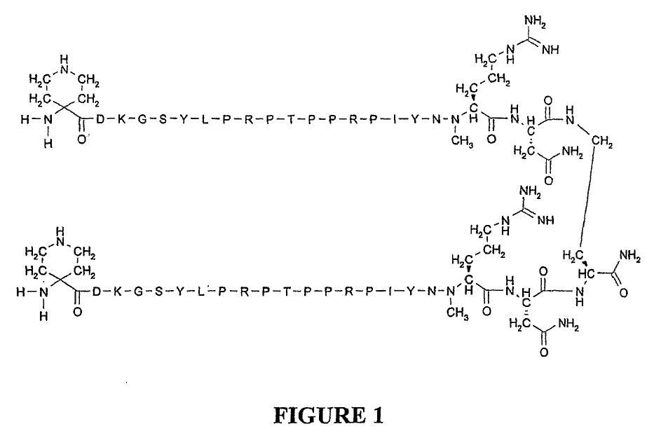

Positive controls for DnaK inhibition can also be provided in the screening method. Known DnaK inhibitors useful as positive controls include DnaK peptide inhibitors, such as, e.g., pyrrhocoricin (Val-Asp-Lys-Gly-Ser-Tyr-Leu-Pro-Arg-Pro-Thr-Pro-Pro-Arg-Pro-Ile-Tyr-Asn-Arg-Asn; SEQ ID NO: 1), drosocin (Gly-Lys-Pro-Arg-Pro-Tyr-Ser-Pro-Arg-Pro-Thr-Ser-His-Pro-Arg-Pro-Ile-Arg-Val; SEQ ID NO: 2) and active analogs thereof. A preferred analog is an analog of pyrrhocoricin termed CHP-105, the structure of which is shown in FIG. 1 (see Cudic et al., Peptides 24:807-820 (2003)).

The DnaK peptide inhibitors can be provided by conventional peptide synthesis or by recombinant DNA means. For example, pyrrhocoricin and various analogs thereof can be provided as described in Otvos, Jr. et al., supra and International Patent Publication No. WO 00/78956.

Follow-on assays can also be performed to confirm the antibacterial activity of any compound identified as a DnaK inhibitor. Identified compounds can first be filtered using either an in vitro or in silico filter to remove inhibitors of e.g., luciferase, thereby allowing the remaining “hits” to be more readily prioritized. Standard cell-based MIC determinations can then be performed in bacteria as a follow-on confirmatory assay. For compounds not readily capable of penetrating the bacterial cell wall/membrane, bacterial strains that have partially compromised cell walls/membranes, such as E. coli D22, can be utilized. A secondary DnaK in vitro screen can also be performed by using an alternative substrate protein than that used in the initial round of identification.

Although the screening methods described herein generally are used as an assay to identify previously unknown DnaK inhibitors that can act as antibacterial agents, the methods can also be used to confirm and/or standardize the desired activity of a known DnaK inhibitor or to optimize the structure and/or activity of a known DnaK inhibitor during structural modification (e.g., derivatives, analogs, etc.).

Specific embodiments of the present invention will now be described in the following Examples. The Examples are illustrative only, and are not intended to limit the remainder of the disclosure in any way.

### EXAMPLES

### Example 1

### Background

The functionality of in vitro refolding assay employing chemically denatured firefly luciferase and E. coli DnaK, GrpE and DnaJ was tested using the DnaK inhibitor CHP-105. The chaperone proteins were obtained from Stressgen Bioreagents Corp., and CHP-105 was prepared as described in Cudic et al., Peptides 23:2071-2083 (2002).

The assay was run in three consecutive stages: 1) chemical denaturation of firefly luciferase, 2) chaperone induced refolding and 3) detection of luciferase levels. Efforts were made to optimize these three steps, with focus on maximizing the detected signal while minimizing the background spontaneous refolding of the luciferase substrate.

### 1) Chemical Denaturation

The following buffers and solutions were prepared:

Denaturing buffer: Guanidinium hydrochloride (6M), Tris-HCl (30 mM), pH 7.4, DTT (5 mM) [*should be replaced every 4 wks due to degradation of the DTT];

Refolding buffer: MOPS (10 mM), pH 7.5, KCl (50 mM), MgCl₂ (5 mM);

Refolding buffer plus: Refolding buffer, DTT (2.5 mM), ATP potassium salt (5 mM), BSA (2.5 mg/mL) [*should be prepared fresh each day to avoid significant degradation of the ATP component]; and

Chaperone protein mixture: DnaK stock solution at 12.86 μM (38.8 μL), DnaJ stock solution at 34.1 μM (3.67 μL), GrpE stock solution at 34.1 μM (27.3 μL) and Refolding buffer (930.2 μL) [*should be routinely prepared fresh each day to avoid freeze/thaw-related degradation of the DnaK component].

To denaturing buffer (24 μL) was added 8 μL of a stock solution of firefly luciferase (25 μM). The sample was mixed thoroughly and allowed to stand for 30 min at room temperature (sample designated D30). In parallel, a positive control sample was prepared from Refolding buffer and the luciferase stock solution (sample designated C30) An initial evaluation of the denaturation stage, reinforced by literature data, suggests that the duration of this stage is not critical and may be adjusted upward from the 30 min window to facilitate plate preparation without adversely affecting the outcome of the assay. However, holding of a denatured sample from run to run or day to day was found to lead to extensive irreversible denaturation.

C30 (5 μL) was added to Refolding buffer plus (995 μL) and mixed thoroughly (sample designated dil C30). D30 (5 μL) added to Refolding buffer plus (995 μL) and mixed thoroughly (sample designated dil D30). Spontaneous refolding was noted to begin immediately upon dilution of D30; therefore, assay plates were prepared during the 30 min denaturation window and the sample used as quickly as possible.

2) Chaperone Induced Refolding

Four sets of assay wells were prepared prior to generating dil D30:

A. Positive control: H₂O (5 mL), Refolding buffer (25 μL);

B. Negative control (spontaneous refolding): H₂O (5 μL), Refolding buffer (25 mL);

C. Chaperone induced refolding: H₂O (5 μL), chaperone protein mixture (25 μL); and

D. Sample mixture: Serially diluted CHP-105 in water (5 μL), chaperone protein mixture (25 μL).

To A (positive control) was added 20 μL of dil C30. To B-D was added 20 μL of dil D30. The four samples were gently mixed and then allowed to stand for 180 min.

3) Detection of Luciferase Signal

To all wells was added 125 μL Refolding buffer, followed by 25 μL of Steady-Glo® luciferase assay substrate (Promega). The samples were mixed gently and then allowed to stand for 2-5 min before reading in a Dynatech ML2200 luminometer. Readouts were expressed as % Refolding compared to the positive control.

Results

Previous reports of DnaK-induced refolding of denatured firefly luciferase had employed DnaK concentrations in the micromolar range, along with equimolar amounts of the two co-chaperone proteins DnaJ and GrpE. Such concentrations would be cost prohibitive in a HTS operation. We observed that increasing the concentration of GrpE above the 1:1:1 level resulted in greater folding at all time points (15-180 min), whereas the equivalent increase in DnaJ concentration only lead to enhanced refolding at later time points (greater than 60 min). On a molar basis, GrpE is considerable cheaper than the other proteins (GrpE:DnaK:DnaJ=1:3.5:7); therefore, we focused on optimizing the concentration and ratio of protein components employing an excess of GrpE. Employing the chaperone proteins at reduced concentrations (DnaK:20 nM, DnaJ:50 nM, GrpE:40 nM) with a 3 hr refolding window reproducibly gave a refolding of ˜20% of the denatured luciferase (compared to ˜5% spontaneous refolding).

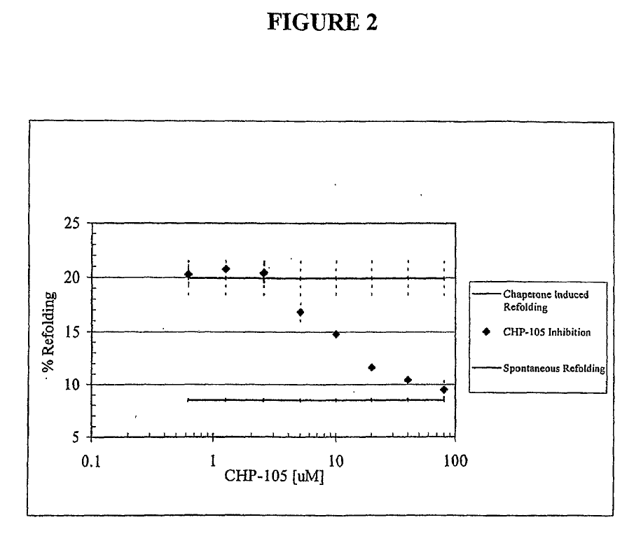

Intended uses of the screening method disclosed herein include the evaluation of pyrrhocoricin peptide analogs as DnaK inhibitors. Initial observations showed that peptide concentrations in the range of 10-50 μM lead to a measurable non-specific enhancement of the background refolding (i.e., in the absence of chaperone proteins), presumably due to hydrophobic stabilization of the denatured luciferase species. This effect was reduced to insignificant levels by the addition of BSA (1 mg/mL) to the refolding mixture. The assay protocol was successfully employed to measure the inhibitory potential of a range of pyrrhocoricin analogs. The dose response curve for CHP-105 is shown in FIG. 2, and is representative of all the data generated to date.

### Example 2

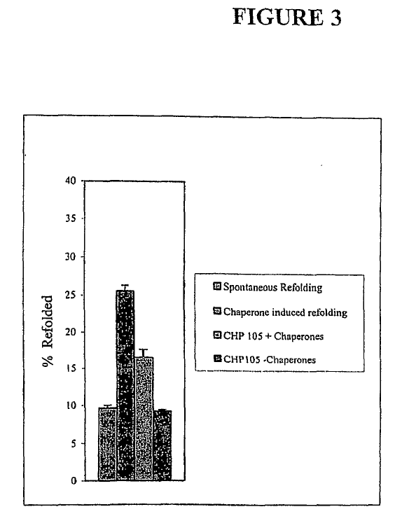

The evaluation of peptides described in Example 1 was performed in strictly aqueous conditions. The evaluation of small organic molecule inhibitors, however, may require the use of organic solvents, such as DMSO, in the refolding reaction. As shown in FIG. 3, 5% DMSO within the refolding mixture had no significant effect on chaperone-induced refolding (second column) or on inhibition of refolding by 10 μM CHP-105 (third column).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the following claims.

All publications cited in the specification, both patent publications and non-patent publications, are indicative of the level of skill of those skilled in the art to which this invention pertains. All these publications are herein fully incorporated by reference to the same extent as if each individual publication were specifically and individually indicated as being incorporated by reference.

What is claimed:

1. A screening method for identification of a DnaK inhibitor, comprising the steps of:
(a) providing a denatured substrate protein in solution;
(b) contacting said substrate protein with DnaK, or a homolog thereof, in the presence and absence of a compound suspecting of having DnaK inhibitory activity; and
(c) determining whether the activity of said substrate protein in the presence of said compound is decreased relative to the activity of said substrate protein in the absence of said compound,
whereby a decrease in substrate protein activity indicates that said compound is a DnaK inhibitor.

2. The screening method of claim 1, wherein the substrate protein is firefly luciferase.

3. The screening method of claim 2, wherein the firefly luciferase is denatured in guanidinium hydrochloride.

4. The screening method of claim 2, wherein the DnaK, or a homolog thereof, is derived from a bacterial genus selected from the group consisting of Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, Bordetella and Francisella.

5. The screening method of claim 1, wherein step (b) further includes DnaJ and GrpE.

6. The screening method of claim 5, wherein the concentration of GrpE is greater than that of DnaK and DnaJ.

7. The screening method of claim 6, wherein the concentration of GrpE is at least about 2-fold greater than that of DnaK and DnaJ.

8. The screening method of claim 7, wherein the concentration of GrpE is about 2-fold greater than that of DnaK.

9. The screening method of claim 7, wherein the concentration of GrpE is about 8-fold greater than that of DnaJ.

10. The screening method of claim 7, wherein the concentration of GrpE is about 2-fold greater than that of DnaK and about 8-fold greater than that of DnaJ.

12. The screening method of claim 1, wherein the compound is a peptide.

13. The screening method of claim 1, wherein the compound is a derivative or analog of pyrrhocoricin.

14. The screening method of claim 1, wherein the compound is a small organic compound.

15. The screening method of claim 14, wherein the small organic compound is dissolved in an organic solvent prior to contacting the substrate protein with DnaK.

16. The screening method of claim 1, further comprising confirming the antibacterial activity of the compound in a bacterial cell-based assay.

17. The screening method of claim 16, wherein the confirmatory assay includes a bacterial strain having a partially compromised cell wall/membrane.

18. The screening method of claim 1, further comprising a positive control for DnaK inhibition.

19. The screening method of claim 18, wherein the positive control is a known peptide inhibitor.

20. The screening method of claim 19, wherein the known peptide inhibitor is pyrrhocoricin or a derivative or an analog thereof.

21. The screening method of claim 20, wherein the analog is CHP-105.

22. The screening method of claim 1, further comprising a negative control for spontaneous refolding.
